(19) United States
(12) Patent Application Publication
(10) Pub. No.: US 2026/0207379 A1
(43) Pub. Date: Jul. 23, 2026

(54) INSTRUMENT FOR OPHTHALMIC SURGERY

(71) Applicant: MORIA, BOURBON-L'ARCHAMBAULT (FR)

(72) Inventor: Jean-Luc AUFAURE, BOURBON-L'ARCHAMBAULT (FR)

(73) Assignee: MORIA, BOURBON-L'ARCHAMBAULT (FR)

(21) Appl. No.: 19/158,199

(22) PCT Filed: Feb. 8, 2024
(86) PCT No.: PCT/EP2024/053122

§ 371 Date: Aug. 20, 2025

(30) Foreign Application Priority Data
Feb. 22, 2023 (FR) FR2301632

(51) Int. Cl. A61F 9/007

(52) U.S. Cl. CPC A61F 9/00727

(57) ABSTRACT

An instrument having a clamp with an active end and a sleeve (2) receiving the sliding clamp; a central body with a first proximal end integral with a handle and a second distal end to which is secured a first end of the clamp, opposite the active end; and an actuator formed by levers extending around the central body and having first ends connected to the central body and second ends joined by a bushing secured to the sleeve. The levers are elastically deformable between a first deactivated position of the clamp in which the active end of the clamp is open and protruding from the sleeve and a second activated position in which the sleeve exerts a force on the active end of the clamp by forcing the closure of the active end of the clamp. The central body, the actuator and the bushing (7) are a single piece.

### BACKGROUND OF THE INVENTION

The present invention relates to the field of instruments used in ophthalmic surgery.

The performance of ophthalmic surgeries and in particular vitreoretinal surgeries requires the use of numerous instruments for single use or that can be autoclaved, intended to visualise, cut, grasp, detach the membranes that form on different parts of the eye during certain pathologies.

Among these, distally controlled retinal forceps are specifically used to grasp the membranes to be removed. In the deactivated state, the clamp is open, the practitioner by actuating the control, generally by simple pressure, will trigger the closure of the clamp. In most of today's retinal forceps, the clamp of the retinal forceps is inserted into a tube with the active part of the clamp protruding from said tube. The opening/closure (deactivation/activation) of the clamp is achieved by the translation of the tube along the clamp. Thus, to close the clamp, the practitioner will exert pressure on the control which will cause the tube to move along the clamp to engage the active part of said clamp and thus close it. The translation takes place in a direction from the proximal end of the instrument to its opposite distal end which comprises the active part of the clamp. To open the clamp, the practitioner releases the pressure exerted on the control causing a reverse movement of the tube to disengage the active end of the clamp (from the distal end to the proximal end of the instrument) and thus cause it to open.

For example, such retinal forceps are described in applications WO-A-2021/038428 or WO-A-2018/156341.

The number of component parts of existing retinal forceps is large and makes the manufacturing process long and expensive. Also, the arrangement of these different parts makes cleaning difficult. Thus, to avoid contamination linked to poor cleaning of the instrument, practitioners prefer single-use retinal forceps. It is therefore necessary to reconcile a sales price for retinal forceps that is compatible with a single use, which is therefore relatively low, even though the production costs related to the large number of component parts and the complexity of the process of assembling said parts are currently high. £ Manufacturers of surgical instruments and in particular of retinal forceps therefore have a constant concern to reduce production costs without sacrificing either the quality or the ergonomics of the retinal forceps.

### OBJECT OF THE INVENTION

A particular aim of the invention is to propose an instrument of retinal forceps-type, the manufacture of which is simplified and less expensive, while preserving its ergonomics.

### SUMMARY OF THE INVENTION

To this end, according to the invention, an instrument is provided comprising:
a clamp with an active end and a sleeve receiving the sliding clamp,
a central body with a first proximal end integral with a handle and a second distal end to which a first end of the clamp is secured, said first end being opposite the active end of the clamp,
an actuator formed by a set of levers extending around the central body and having first ends connected to the central body and second ends joined by a bushing to which the sleeve is secured, the levers being elastically deformable between a first deactivated position of the clamp in which the active end of the clamp is open and protruding from the sleeve and a second activated position in which the sleeve exerts a force on the active end of the clamp so as to tighten and force the closure of the active end of the clamp. According to the invention, the central body, the actuator and the bushing of the instrument are a single piece.

Thus, the instrument according to the invention comprises a very limited number of parts, easy and economical to produce and assemble while maintaining excellent ergonomics of use for the practitioner.

The instrument according to the invention may further comprise a guide nose slidably receiving at least a part of the sleeve to be guided therein, said guide nose being fixed and connected to the central body. In preferred manner, the guide nose is connected to the central body by arms passing between the levers. Said guide nose may also comprise a housing receiving the sliding bushing.

Advantageously, the guide nose, the central body, the actuator and the bushing of the instrument according to the invention are a single piece.

In addition, the central body and the handle of the instrument may also be a single piece.

The invention also provides a method of manufacturing an instrument according to the invention comprising the steps of
a. manufacturing in additive manufacturing a single piece comprising at least, a central body, an actuator, a bushing and a guide nose;
b. securing the sleeve in the bushing;
c. slidably inserting the clamp into the sleeve and securing it in the central body.

Additive manufacturing makes it possible to obtain the instrument according to the invention, quickly and inexpensively.

Other features and advantages of the invention will emerge upon reading the description below of a particular and non-limiting embodiment of the invention.

### BRIEF DESCRIPTION OF THE DRAWINGS

Reference will be made to the accompanying drawings, among which:

FIG. 1: a slightly perspective view of an instrument according to the invention, the instrument being deactivated, the clamp in the open position;

FIG. 2: view in longitudinal section along the axis X of an instrument according to the invention, the clamp being in the open position and presented at 90° with respect to FIG. 1.

FIG. 3: longitudinal sectional view along the axis X of an instrument according to the invention, the clamp being in the open position.

FIG. 4: enlarged view along IV showing the distal part of the central body, the bushing, the clamp, the sleeve and the guide nose.

FIG. 5: enlarged view along V showing the distal part of the central body, the bushing, the clamp, the sleeve and the guide nose.

FIG. 6: longitudinal sectional view along the axis X of an instrument according to the invention, the clamp being in the closed position.

FIG. 7: enlarged view according to VII showing the distal part of the central body, the bushing, the clamp, the sleeve and the guide nose when the clamp is in the closed position.

### DETAILED DESCRIPTION OF THE INVENTION

In the present text, the elements qualified as “proximal” are the elements closest to the practitioner's hand. On the other hand, the elements described as “distal” are the elements furthest from the practitioner's hand.

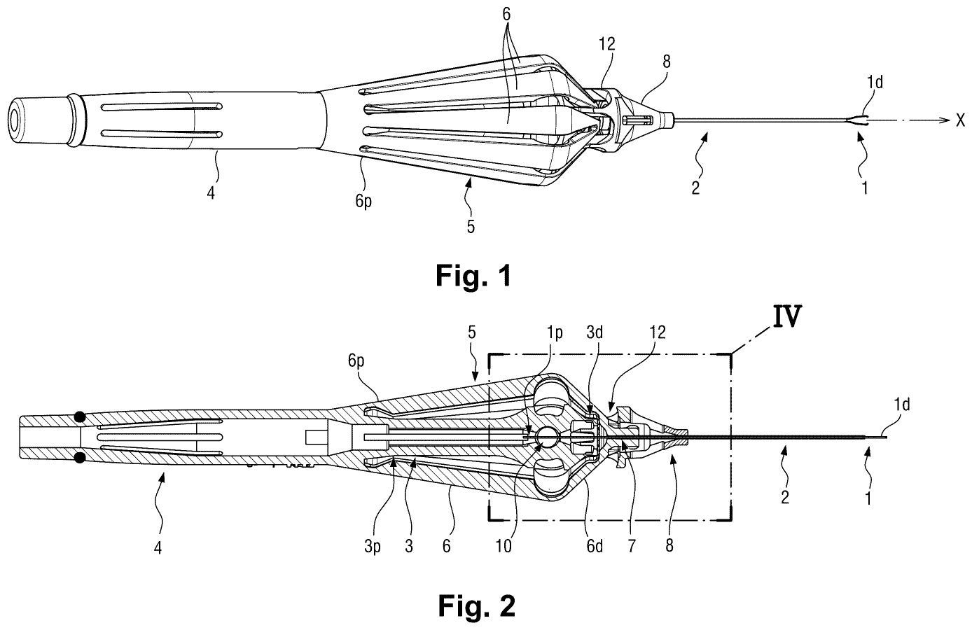

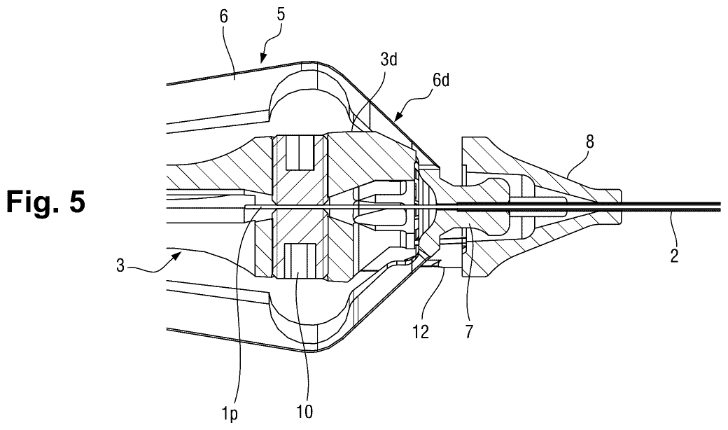

With reference to FIGS. 1 to 7 and according a particular embodiment, the present invention relates to an instrument, in particular a retina forceps, comprising a rod-shaped clamp 1 with an active distal end 1d and a proximal end 1p intended to be secured in a central body 3.

The clamp 1 defines a longitudinal axis X of the instrument passing through the distal end 1d of the clamp 1 and the proximal end 1p of the clamp 1.

The active distal end 1d is separated into two elastically deformable branches terminated by a gripping surface for grasping a tissue fragment.

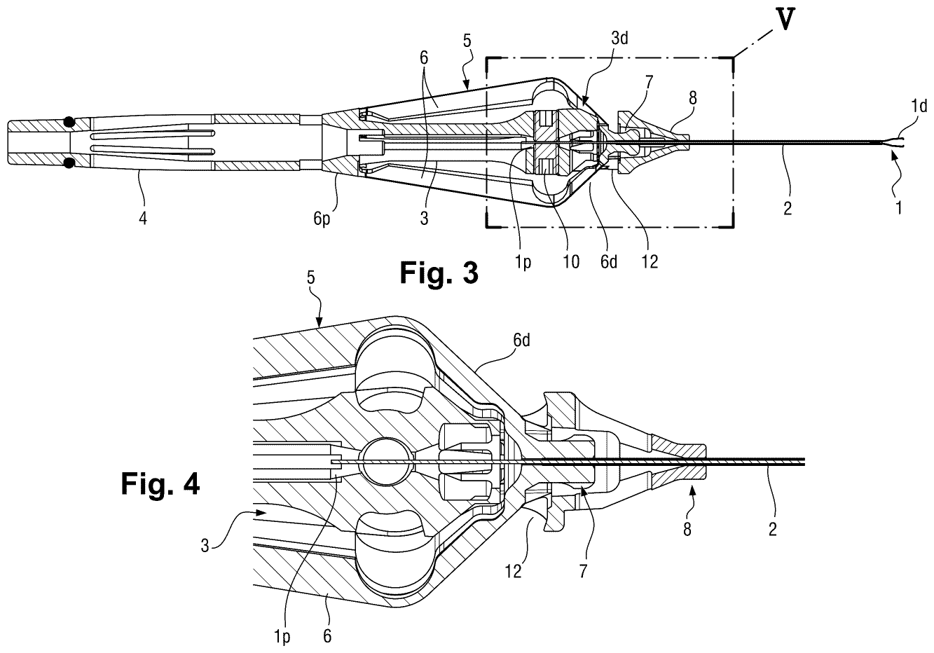

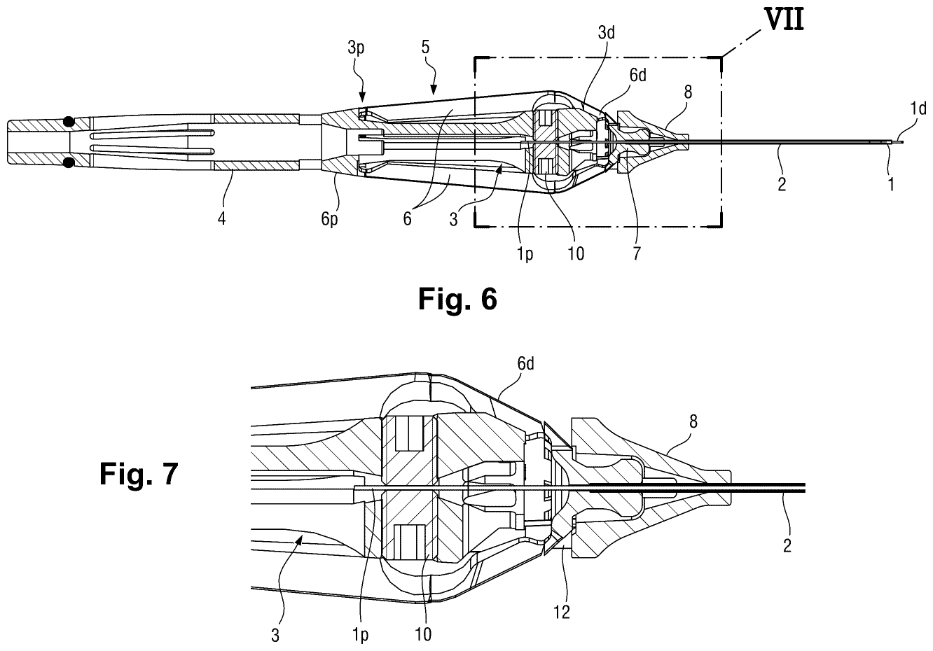

In the deactivated position, the two branches of the distal end 1d of the clamp 1 are in a rest state in which they are spaced apart from each other, as is particularly illustrated in FIGS. 1 and 3. In the activated position, the two branches of the distal end 1d of the clamp 1 are in a state of elastic deformation in which they are in contact with each other in such a way that the gripping surfaces allow the tissue fragment to be grasped, as is particularly illustrated in FIG. 6. The person skilled in the art will know how to choose the clamp 1 as a function of the end use of the instrument that is the object of the invention.

The instrument according to the invention also comprises a sleeve 2, in the form of a hollow cylindrical tube with an internal diameter slightly greater than that of the clamp 1, in which the clamp 1 is slidably engaged. The diameter is defined in such a way that the clamp 1, once engaged in the sleeve, can slide inside the sleeve between an activated position and a deactivated position. In the activated position, the sleeve 2 makes it possible to exert a tightening force on the two branches of the distal end 1d to bring the gripping surfaces of the branches into contact and thus close or in other words activate the clamp 1.

The clamp 1 and the sleeve 2 are made of a biocompatible and sterilisable material such as, by way of non-limiting examples, stainless steel or a chromium-cobalt alloy.

The instrument of the invention further comprises a central body 3, here substantially in the form of a hollow tube. The central body 3 has a distal part 3d, here domed, to which the proximal end 1p is secured. The proximal end 1p of the clamp 1 is secured in the distal part 3d of the central body 3 by at least one screw 10. Here, the proximal end 1p is secured by two screws 10 that are engaged transversely to the longitudinal axis X in the distal part 3d, in opposition to each other, so as to clamp the proximal end 1p between them. Thus, the proximal end 1p of the clamp 1 is held inside the central body 3 between the two screws 10 as in a vice. The person skilled in the art can thus adjust the holding of the clamp 1, possibly changing it by tightening/loosening at least one of the two screws 10. The person skilled in the art is able to define the dimensions of the central body 3 and the fixing means of the clamp 1 as a function of the material used and the manufacturing constraints. By way of illustration, here the central body is made of Pa12 (Polyamide 12) with a diameter of approximately 12 mm in the widest part of the central body and a length of the order of 35 to 40 mm, preferably 38 mm.

The central body 3 therefore has a proximal end 3p, opposite the distal end 3d, integral with a handle 4 which forms an extension thereof so as to facilitate the manipulation of the instrument by the practitioner's hand. The handle 4 is also approximately cylindrical in shape. The person skilled in the art will know how to determine the length and the diameter of the handle as a function of the desired ergonomics. By way of illustration, the length of the handle is between 50 and 150 mm and the diameter between 7 and 10 mm.

The instrument of the invention also comprises an actuator 5 formed by a set of levers 6 extending around the central body 3 and having first proximal ends 6p connected to the central body 3 and second distal ends 6d which extend beyond the distal portion 3d and which are joined by a bushing 7 extending in front of the distal part 3d and coaxially therewith. The sleeve 2 is secured to the bushing 7 by any means, for example by gluing, welding, or simply holding it in place. The levers 6 are elastically deformable between a first deactivated position of the clamp 1 in which the active end 1d of the clamp 1 is open and protrudes from the sleeve 2, and a second activated position in which the sleeve 2 covers and tightens the active end 1d of the clamp 1 so as to force the closure of the active end of the clamp. Each lever 6 comprises a proximal segment and a distal segment which are connected together by a first flexible blade defining an open angle of less than 180° when the actuator 5 is in the deactivated position, a second flexible blade connecting the proximal segment to the central body 3 and a third flexible blade connecting the distal segment to the bushing 7. The segments and the flexible blades are a single piece. In the activated position, as particularly illustrated by FIG. 6, the two segments are substantially aligned with each other, the angle formed between the two segments then being substantially equal to 180°. The person skilled in the art will know how to determine the respective length of each of the segments of the blade as well as the value of the angle formed as a function of the length of the central body 3 and the length of the travel of the bushing 7 so that, in the activated position, the distal end of the sleeve 2 closes the active distal end 1d of the clamp 1 without covering the gripping surfaces of said distal end 1d. Obviously, the person skilled in the art will also know how to take into account the nature of the material used in the sizing of the levers and determine the number of levers 6 necessary to form the actuator 5 as a function of the dimensions of each lever, the material used and the desired ergonomics.

By way of illustration, the actuator 5 may comprise ten levers 6 made of polyamide 12 (Pa12); each lever 6 has a variable width ranging from 1 to 5 mm for the distal segment, and from 2 to 5 mm for the proximal segment close to the handle 4. The thinnest end of the lever segments corresponding respectively for the end 6p to that which is close to the proximal end 3p of the body and for the end 6d to that which is in the extension of the bushing 7. The widest part of the segments lying between the two and being intended to accommodate the practitioner's fingers when using the instrument. The proximal segment and the distal segment of each lever 6 here have a respective length of about 30 mm and less than 10 mm and form an angle of about 130° when the actuator 5 is deactivated.

The proximal ends 6p of the levers 6 are here in one piece with the proximal part 3p of the central body 3. On the other hand, the distal ends 6d of the levers 6 are in one piece with the bushing 7 which is movable in translation relative to the central body 3 along the axis X. Thus, to activate the clamp, the manipulator will exert pressure on all or part of the levers 6 of the actuator 5, preferably on their widest part, the levers 6 will unfold until the segments of each lever 6 form between them an angle substantially equal to 180° by imparting a translatory movement to the bushing 7 and therefore to the sleeve 2 which is secured thereto along the axis x in the direction of the distal end 1d. The distal end of the sleeve 2 will then exert pressure on the two branches of the distal end 1d of the clamp 1 and cause these two branches to move closer together until the clamp 1 is closed or in other words activated. Conversely, when the practitioner releases the pressure exerted on the actuator 5, the levers 6 will fold under the effect of their elasticity until the two segments form the initial angle between them. The bushing 7 and the sleeve 2 will perform a reverse translation towards the handle 4: the two branches of the distal end 1d of the clamp 1 are then released from the distal end of the sleeve 2 and move apart from each other under the effect of their elasticity. The travel of the sleeve 2 is stopped when the levers 6 are in their rest state and the bushing 7 is in the vicinity of the distal end 3d of the central body 3.

According to the invention, the central body 3, the actuator 5 and the bushing 7 of the instrument are a single piece. The central body 3, actuator 5 assembly composed of the set of levers 6 joined by their distal end to form the bushing 7 are made by additive manufacturing. For example, the central body, the actuator, and the bushing may be manufactured using an EOS brand printer fed with Polyamide 12 (Pa12), the manufacture being carried out in successive layers along the X axis.

In the preferred embodiment of the invention described here, the instrument according to the invention comprises a fixed guide nose 8, connected to the central body 3, which extends in front of the bushing 7 and covers part of the latter. Preferably, the guide nose 8 is connected to the distal end of the central body 3 by arms 12 passing between the levers 6. The arms 12 are here in the extension of the distal part 3d of the central body 3. The guide nose 8 comprises a drilling that is collinear with the axis X and that slidably receives a part of the sleeve 2 so as to guide said part of the sleeve 2 and limit the risk of bending the sleeve 2. The diameter of the drilling is slightly greater than the diameter of the sleeve 2 so that the latter can slide freely in the guide nose 8. The guide nose 8 is here substantially in the shape of a hollow truncated cone and drilled at its top. The guide nose 8 further comprises a rear housing freely receiving a part of the bushing 7, in particular for the activated position. Thus, in the activated position of the instrument, the bushing 7 comes close to the inner wall of the housing provided inside the guide nose 8, while in the deactivated position of the instrument, the bushing 7 is close to the distal end of the central body 3. The travel of the bushing 7, and consequently of the sleeve 2, is therefore between, on the one hand, the distal end of the central body 3 and, on the other hand, the internal wall of the housing of the guide nose 8. It should be noted that the guide nose is fixed. The presence of a guide nose 8 makes it possible to improve the accuracy of the instrument between two extreme positions. It also makes it easier to slide the sleeve 2 along the clamp 1 by stiffening its hold, thus improving the accuracy of the instrument. In addition, since the guide nose is fixed and integral with the central body 3, the distance between the end 1d of the clamp and the top of the guide nose 8 remains constant, whether the instrument is activated or not. The useful length of the clamp 1—sleeve 2 assembly which penetrates into the eye is thus totally preserved. This allows the practitioner to manipulate the instrument in the same manner whether it is activated or not.

In accordance with the invention, the guide nose 8, the central body 3, the actuator 5 and the bushing 7 are manufactured in the form of an integral single piece. The central body 3, actuator 5 assembly composed of the set of levers 6 joined by their distal end to form the bushing 7, the bushing 7 itself and the guide nose 8 the arms 12 of which pass between the levers 6 and are in the extension of the distal part of the central body 3 is made in additive manufacturing.

Preferably, the handle 4 and the central body 3 form a single piece.

Thus, the instrument according to the invention comprises only three distinct elements:
a clamp 1
a sleeve 2
a single and integral piece comprising a central body 3, an actuator 5, a bushing 7, and optionally a guide nose 8 and a handle 4.

Manufacturing costs are therefore greatly reduced since it suffices to manufacture the single piece by additive manufacturing, then to secure the sleeve 2 in the bushing 7, and finally to slide the clamp 1 into the sleeve 2 and secure it in the central body 3, all the more so when the single piece also comprises the guide nose 8 and/or the handle 4. The invention also relates to such a manufacturing method.

The sleeve 2 is secured manually or automatically. The sleeve 2 is inserted into the drilling of the guide nose 8 and then into the drilling of the bushing 7 where it is secured. Finally, once the sleeve 2 has been secured in the bushing 7, the clamp 1 is slidably inserted into the sleeve 2 until its proximal end 1p reaches the fixing means 10 connected to the central body 3. Once the clamp 1 is correctly positioned, in the central body 3, the fixing means, in this case the screws 10, are tightened so as to secure the clamp 1 and prevent any translational movement thereof along the axis X.

The instrument in accordance with the invention therefore comprises few parts, which simplifies assembly and reduces manufacturing costs. The use of additive manufacturing makes it possible to produce shapes that are difficult or impossible to obtain with other technologies: for example, it makes it possible to produce an instrument according to the invention with a fixed guide nose 8 integral with the central body 3. This manufacturing time, in particular reduced assembly time and the use of additive manufacturing make it possible to produce instruments easily at a very low cost while maintaining the ergonomics and the accuracy of the instrument.

Naturally, the invention is not limited to the embodiment described, but covers any variation entering into the scope of the invention, such as defined by the claims.

In particular, the clamp is held in the central body by two screws, but the proximal end of the clamp may also be held by a single screw. The proximal end of the clamp can alternatively be glued or welded into the central body.

1. An instrument comprising:
a. a clamp with an active end and a sleeve receiving the sliding clamp
b. a central body with a first proximal end integral with a handle and a second distal end to which is secured a first end of the clamp, opposite the active end of said clamp,
c. an actuator formed by a set of levers extending around the central body and having first ends connected to the central body and second ends joined by a bushing to which the sleeve is secured, the levers being elastically deformable between a first deactivated position of the clamp in which the active end of the clamp is open and protruding from the sleeve and a second activated position in which the sleeve exerts a force on the active end of the clamp by forcing the closure of the active end of the clamp
wherein the central body, the actuator and the bushing are a single piece, said instrument comprising a guide nose through which at least a part of the sleeve passes to be guided therein, said guide nose being fixed and connected to the central body.

2. The instrument according to claim 1, wherein the guide nose is connected to the central body by arms passing between the levers.

3. The instrument according to claim 1 wherein the guide nose comprises a housing receiving the bushing freely.

4. The instrument according to claim 1 wherein the guide nose the central body, the actuator and the bushing are a single piece.

5. The instrument according to claim 1 wherein the handle and the central body form a single piece.

6. A method of manufacturing an instrument according to claim comprising the steps of:
a. manufacturing in additive manufacturing a single piece comprising at least, a central body, an actuator, a bushing and a guide nose;
b. securing the sleeve in the bushing;
slidably inserting the clamp into the sleeve and securing it in the central body.
